(19) United States
(12) Patent Application Publication
(10) Pub. No.: US 2006/0085011 A1
(43) Pub. Date: Apr. 20, 2006

(54) Instrument system for the insertion of intervertebral disk implants

(75) Inventors: Michael Filippi, Schaffhausen (CH); Guido Casutt, Sulz-Rickenbach (CH); Jochen Reinmuth, Winterthur (CH); Mathias Heller, Raeterschen (CH)

Correspondence Address: TOWNSEND AND TOWNSEND AND CREW LLP, TWO EMBARACADERO CENTER, 8TH FLOOR, SAN FRANCISCO, CA, 94111, US

(73) Assignee: Zimmer GmbH, Winterthur (CH)

(21) Appl. No.: 11/250,317

(22) Filed: Oct. 13, 2005

(30) Foreign Application Priority Data
Oct. 15, 2004 (EP) 04024653.0

(51) Int. Cl. A61F 2/34

(52) U.S. Cl. 606/99

(57) ABSTRACT

The invention relates to an instrument system for the insertion of intervertebral disk implants including two implant plates and one implant core comprising at least one trial unit including two trial elements of preferably plate shape for the determination of an implant suitable for the respective patient, at least one machining unit for the preparation of the surfaces of the bodies of the vertebra of the patient which includes operating elements having two sections of preferably forked shape and at least one holding unit which can be coupled to the implant plates for the insertion of the implant, with the trial unit, the machining unit and the holding unit each being able to be coupled to a traction instrument in particular in the manner of forceps for the pressing apart of the trial elements, the operating elements or the implant elements.

### CROSS-REFERENCE TO RELATED APPLICATIONS

This application claims the benefit of European Patent Application No. EP 04 024 653.0, filed on Oct. 15, 2004. The disclosure of the above application is incorporated herein by reference.

### FIELD AND BACKGROUND OF THE INVENTION

The invention relates to an instrument system for the insertion of intervertebral disk implants comprising two implant plates and one implant core.

In operations of this type, the selection of the correct intervertebral disk implant which is optimum for the respective patient is of decisive importance. The selection of the implant takes place preoperatively for the greater part, e.g. using X-ray patterns. However, all the parameters of the implant to be inserted cannot always be determined preoperatively. The optimum height of the implant can in particular be determined in the most suitable manner intraoperatively. Traction instruments are required for this purpose with which the two adjacent bodies of the vertebra between which the intervertebral disk implant is to be inserted can be spread apart. An instrument system for the insertion of intervertebral disk implants should therefore provide the surgeon with the possibility of effecting a spreading apart of the bodies of the vertebra in question for the selection of the suitable implant. At the same time, the set of instruments required for the surgery in total must be compact and easy to handle. This is above all of importance since the selected implant cannot be inserted immediately, but rather a preparation of the respective disk space is previously necessary. The set of instruments should furthermore require as small a space as possible so as not to unnecessarily reduce the available operating space.

### SUMMARY OF THE INVENTION

It is the object of the invention to provide an instrument system of the initially named kind which satisfies the aforesaid demands, i.e. which is compact, has a structure which is as simple as possible and is as easy as possible to handle for the surgeon, while having a reliable function, and which hinders the surgeon as little as possible in each of the individual steps of the operation.

This object is satisfied in accordance with the invention by the features of claim 1 and in particular in that the instrument system includes at least one trial unit including two trial sections of preferably plate shape for the determination of an implant suitable for the respective patient, at least one machining unit for the preparation of the surfaces of the bodies of the vertebra of the patient which includes two operating elements having sections which are preferably of fork shape, and at least one holding unit which can be coupled to the implant plates for the insertion of the implant, with the trial unit, the machining unit and the holding unit being able to be coupled in each case to a traction instrument, in particular of the forceps type, for the pressing apart of the trial sections, the operating elements and the implant plates.

The set of instruments in accordance with the invention permits the carrying out of all necessary surgical steps, namely a final, intraoperative selection of the implant, the subsequent machining of the bodies of the vertebra and the insertion of the selected implant into the prepared disk space. In each of these phases of the operation, the instrument unit used, i.e. the trial unit, the machining unit or the holding unit can be pressed apart by means of a traction instrument. The possibility therefore exists in the trial phase or selection phase to carry out the final determination of the suitable implant with a spreading apart of the corresponding bodies of the vertebra in accordance with the natural shape of a healthy spine. The preparation of the surfaces of the bodies of the vertebra can take place under those conditions under which the final intraoperative selection of the implant was carried out thanks to the possibility of pressing apart the operating element of the operating unit by means of a traction instrument. The bodies of the vertebra are also spread apart on the insertion of the implant in that the implant plates which are coupled to the holding unit and which were previously introduced into the prepared disk space are pressed apart by means of a traction instrument such that the implant core can be introduced without problem between the implant plates. In accordance with the invention, the trial unit, the machining unit and the holding unit are thus based on the same operating principle, namely on the pressing apart of the components of the instrument system or of the implant being used in the respective phase of the operation. The units can be designed due to this common traction principle such that the surgeon only requires one single traction instrument which can, optionally, be coupled to the units by means of corresponding adapter elements.

The trial unit, the machining unit and the holding unit are preferably each provided with coupling sections for the traction instrument at their rear side remote from the surgical area during surgery. The space requirements in directions perpendicular to the anterior-posterior direction can hereby be kept to a minimum since the surgeon can so-to-say remain “in the shadow” of the implant with the instrument system or the traction instrument during the surgery.

In a particularly preferred practical further development of the invention, the trial unit has an adjustable depth gage for the fixing of a reference position for the implant in the anterior-posterior direction.

The circumstance that the selection of an implant ideally suitable for the respective patient is also dependent on the depth, i.e. on the position in the anterior-posterior direction in which the implant is to be inserted, can hereby be taken into account and care can be taken that implant plates are used which have as large an area as possible. With comparatively small bodies of the vertebra, it can thus be attempted to utilize the existing depth of the bodies of the vertebra as much as possible and to introduce the implant plates as deeply as possible so that the center of rotation is disposed as far to posterior as possible. In contrast, it can be indicated, for example with comparatively wide bodies of the vertebra with a relatively low depth, to use implant plates with dimensions which are as large as possible in the anterior-posterior direction to cover an area which is as large as possible, said implant pates only being introduced in a depth which is comparatively low and their end facing to anterior consequently being disposed comparatively closely to the anterior edge of the respective body of the vertebra. The optimum position of the implant in the anterior-posterior direction in the respective case can, in accordance with the invention, be set with the help of the adjustable depth gage provided at the trial unit. The anterior-posterior setting hereby obtained, which is also briefly called the AP position in the following, can then serve as a reference for the further steps of the operation.

It is furthermore proposed in accordance with the invention that the machining unit and/or the holding unit likewise have an adjustable depth gage which is adjustable in accordance with the reference setting of the trial unit. The machining of the surfaces of the bodies of the vertebra and the insertion of the implant plates can thus take place in a manner matched to the selected implant with the help of the reference position initially determined. It is in particular ensured that the machining unit and the holding unit each adopt the correct AP position. It can in particular be ensured by the depth gage that the respectively set AP position cannot change independently during the machining of the surfaces of the bodies of the vertebra or on the insertion of the implant plates.

All depth gages are preferably made at least substantially in the same construction. The instrument system is hereby further simplified.

The depth gage is preferably made at least regionally in the manner of an adjusting screw adjustable relative to the trial unit in the anterior-posterior direction. A rotary movement applied, for example, by means of a screwdriver about an axis extending parallel to the anterior-posterior direction can hereby be converted into a translatory movement of the depth gage or of a section of the depth gage in an anterior-posterior direction relative to the trial unit.

In a preferred practical embodiment, the depth gage is coupled to the trial unit via a pin and groove guide. The adjustment movement can, in this manner, be achieved by a compulsory guidance of the pin in the groove.

The depth gage preferably has an adjustment section, in particular an adjustment section which is rotatable by means of a screwdriver about an axis extending substantially parallel to the anterior-posterior direction, at which an adjustment groove, which extends at least regionally in the manner of a helix or a screw around the axis of rotation, is made for a guide pin arranged at the trial unit.

The shape of the adjustment groove can generally be any desired one. The adjustment groove can thus, for example, be made as a helix with a constant pitch. Alternatively, it is, however, also possible for the adjustment groove to include a plurality of sequential groove sections with different pitches.

It is furthermore proposed that the adjustment groove have a plurality of latch recesses for the guide pin which are spaced apart from one another and correspond to pre-determined depth positions. The depth gage can hereby adopt a plurality of discrete depth configurations or reference configurations which are stable and cannot be released accidentally.

In accordance with a further particularly preferred practical embodiment of the invention, the machining unit includes—in addition to the operating element—at least one guide sleeve for an operating instrument, in particular a cutter or drill, with the guide sleeve being able to be coupled to the machining unit for the fixing of a machining position. The coupling preferably takes place such that the guide sleeve is held tight in directions perpendicular to the anterior-posterior direction with respect to the machining unit.

The machining of the surfaces of the bodies of the vertebra in a defined vertical position pre-determinable by the operating elements is hereby ensured.

The guide sleeve is preferably made both for caudal and for cranial machining and can be coupled for this purpose first with the one operating element and then with the other operating element. The provision of two different guide sleeves for the caudal machining and for the cranial machining of the bodies of the vertebra is not necessary due to this universal usability of the guide sleeve.

Provision can furthermore be made in accordance with the invention for the machining unit to regionally bound a tunnel-like receiving space into which the guide sleeve can be inserted at least partly in the anterior-posterior direction.

In accordance with a further preferred embodiment, the machining unit includes a separate latching element with which a spacing between the operating elements established by means of the traction instrument can be fixed. The latching element hereby takes over the maintenance of the tractioned state such that the traction instrument can be removed while maintaining the set spacing between the operating elements and the machining of the bodies of the vertebra can be carried out without the surgeon being impeded in any way.

The latching element preferably has at least one latching section pushable between the operating elements in the anterior-posterior direction and having a stepped vertical section or one extending in a stair-like manner. The vertical setting at the machining unit, i.e. the spreading open of the bodies of the vertebra by means of the operating elements can hereby take place in discrete steps in that the latching element is subsequently pushed in during the pressing apart of the operating elements as soon as the spacing between the operating elements is large enough to bring the next step of the vertical profile between the operating elements in a latching manner.

The latching section is preferably fork-shaped and includes two latching arms which have identical vertical sections and which can each be pushed between longitudinal edge sections facing one another of the operating elements preferably made in the manner of half-shells or troughs in this region.

Further preferred embodiments of the invention are also recited in the dependent claims, in the description and in the drawing.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIGS. 1-4 illustrate different views of a depth gage in accordance with an embodiment of the invention;

FIG. 5 is a perspective view of a trial unit in accordance with an embodiment of the invention having two depth gauges in accordance with the invention;

FIG. 6 illustrates the trial unit of FIG. 5 in a side view with an introduced trial body;

FIG. 7 illustrates a depth gage in accordance with a further embodiment of the invention;

FIG. 8 is the depth gage of FIG. 7 at a component of a holding unit in accordance with the invention;

FIGS. 9-11 illustrate components of a machining unit in accordance with an embodiment of the invention;

FIG. 12 illustrates the components of FIGS. 9-11 in the assembled state with two depth gages in accordance with the invention;

FIG. 13 illustrates a guide sleeve of the machining device of FIG. 12;

FIG. 14 illustrates a machining unit in accordance with a further embodiment of the invention in the assembled state with two depth gages in accordance with FIG. 7 and a machining instrument;

FIG. 15 illustrates a view from anterior of the machining unit of FIG. 14 without a machining instrument;

FIG. 16 is a perspective view of a holding unit in accordance with a further embodiment of the invention;

FIG. 17 illustrates the holding unit of FIG. 16 in a side view with introduced implant plates;

FIG. 18 illustrates an implant core of an implant insertable by means of the invention with a holding instrument;

FIG. 19 illustrates a further holding instrument for an implant core; and

FIG. 20 illustrates extension elements plugged onto a front part of an only partly shown pair of traction forceps for the support of operating elements of the machining unit of FIGS. 14 and 15.

### DETAILED DESCRIPTION OF THE PREFERRED EMBODIMENTS

The following description of the preferred embodiments is merely exemplary in nature and is in no way intended to limit the invention, its application, or uses.

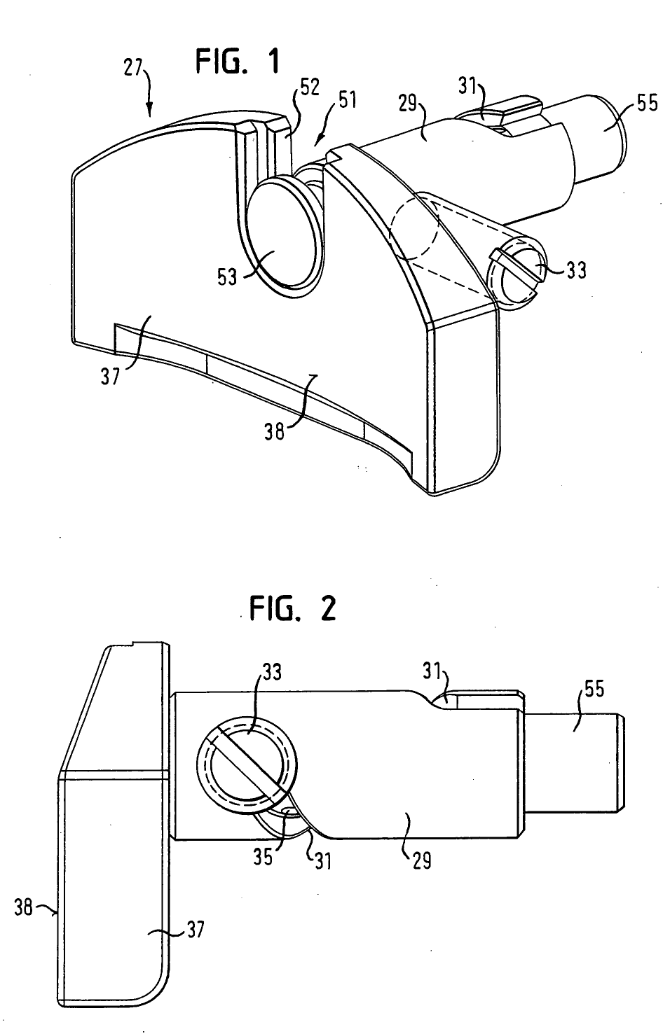

The depth gage 27 in accordance with the invention shown in FIGS. 1 to 4 includes an abutment plate 37 with an abutment surface 38 extending perpendicular to the anterior-posterior direction during surgery and facing posterior. The parameter “anterior-posterior” will also be simply abbreviated to “AP” in the following.

The provision of an abutment plate of this type is not compulsory in accordance with the invention. Alternatively, the end face facing posterior of an adjustment member which is e.g. likewise cylindrical and corresponds to the adjustment section 29 described in the following can serve as an abutment surface and a separate abutment plate can thus be omitted.

The abutment plate 37 is provided with a U-shaped cut-out 51 into which a cylindrical adjustment section 29 having a latched head 53 is introduced. In the introduced state, the plate-shaped latching head 53 engages behind a latching section 52 bounding the cut-out 51 regionally. The adjustment cylinder 29 is hereby fixedly connected to the abutment plate 37 with respect to the AP direction, with the support of the latching head 53 in the cut-out 51, however, permitting a rotation of the adjustment cylinder 29 with respect to the abutment plate 37.

A helical adjustment groove 31 is formed in the outer wall of the adjustment cylinder 29 and a guide pin 33 with an adjustment prolongation is guided therein in a compulsory manner during use. During use, the guide pin 33 is fixedly arranged with respect to the AP direction at the respective unit relative to which the depth gage 27 is to be adjusted, such that a rotation of the adjustment cylinder 29 is converted into a translatory movement in the AP direction by the groove 31 coupled to the pin 33.

Instead of the guide pin 33, a leaf spring provided with a pin projecting into the adjustment groove 31 can, for example, also be provided which is fixedly connected to the respective unit in a suitable manner.

For the rotary actuation of the adjustment cylinder 29, it is provided with an actuation section 55 which faces anterior and onto which a special screwdriver is set which will be considered in more detail at another point.

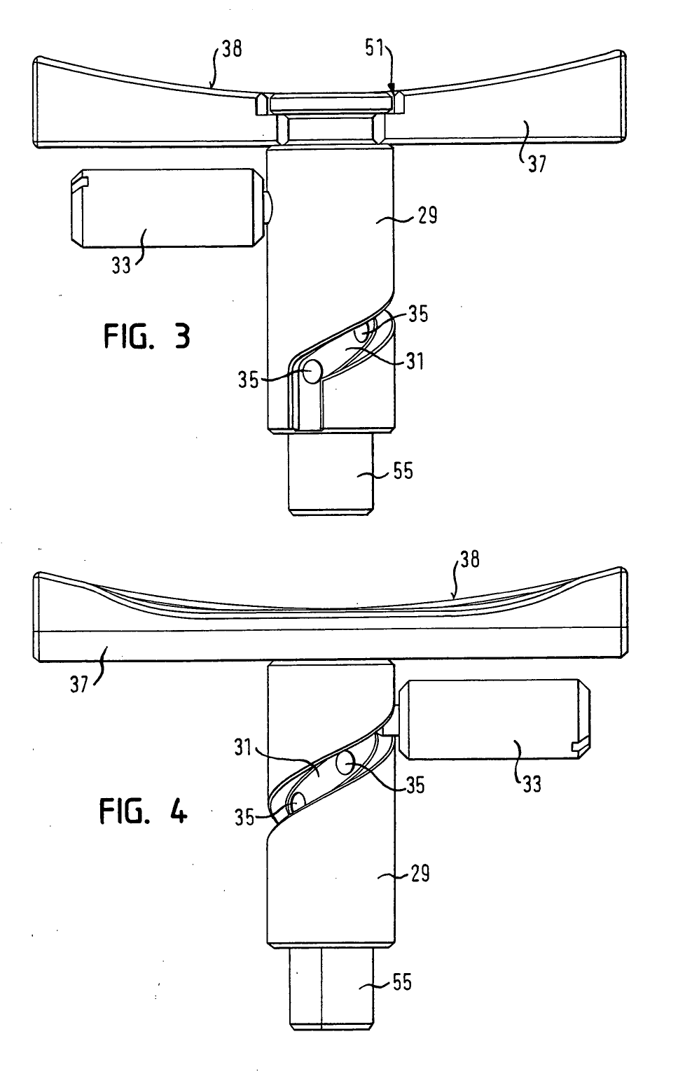

It can in particular be seen from the side view in FIG. 2 that the abutment plate 37 is, for instance, chamfered to anterior in the upper third, i.e. in the region of the cut-out 51 (FIG. 1). FIGS. 3 and 4 in particular show that the abutment surface 38 is concavely curved to match the shape of the bodies of the vertebra.

As FIGS. 2, 3 and 4 show, a plurality of recesses 35 arranged spaced apart along the course of the groove are formed in the base of the adjustment groove 31. The adjustment prolongation of the guide pin 33 projecting into the adjustment groove 31 is biased in the direction of the groove base by means of a compression spring such that it latches into the recesses 35 when the corresponding rotary position of the adjustment cylinder 29 has been reached. The latch connection is made such that the latch positions can be overridden without an additional measure by means of the screwdriver engaging at the actuation section 55, but are nevertheless noticeable for the surgeon so that the reaching of the respective depth positions is signaled to the surgeon by the latch connections.

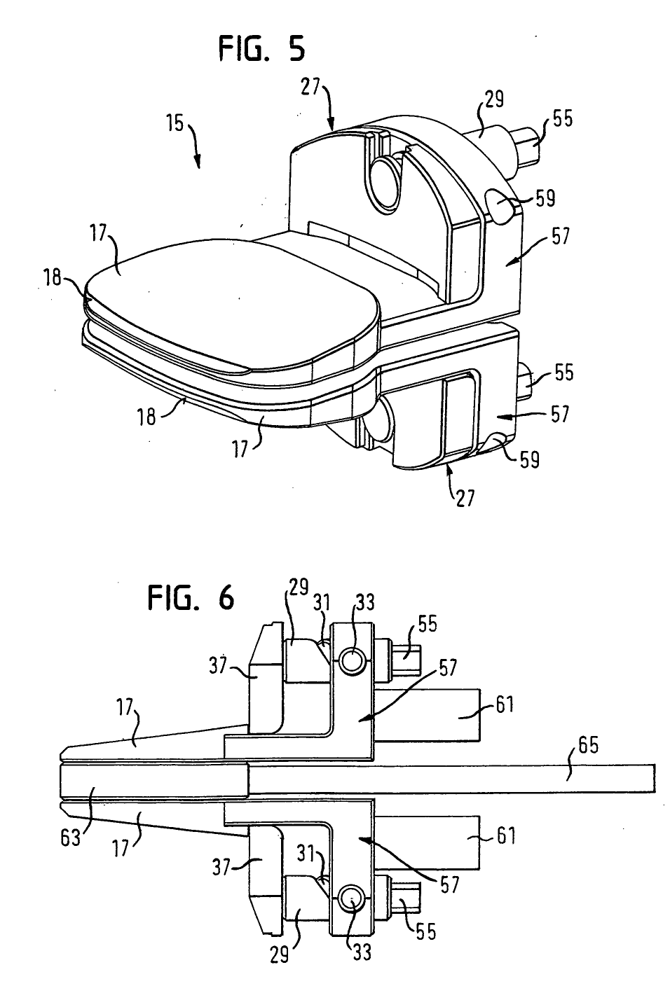

FIGS. 5 and 6 show two depth gages 27 in accordance with the invention in the state attached to a trial unit 15 of the instrument system in accordance with the invention.

The trial unit 15 includes two angular elements 57 of substantially the same construction. The angular section extending in the AP direction is made as a plate-shaped trial section 17. The side of the trial plate 17 facing caudal or cranial is inclined with respect to the AP direction in accordance with an angle determined preoperatively as part of the surgery planning. A set of angular elements 57 with different inclinations of the trial plate 17 is available for each implant size.

It has been found that it is sufficient for practice for only two trial plates 17 of differently pronounced inclination to be kept in stock per implant size both for the upper or cranial part and for the lower or caudal part of the trial unit 15. For the one trial plate, the angle of inclination amounts to 2°, whereas the angle of inclination of the other trial plate amounts to 7°. The total angle between the two inclined sides of the trial plates 17 which can be achieved by the possible combinations of the upper and lower trial plates 17, namely total angles of 4°, 9° and 14°, have proved sufficient for practice. The number of the trial units 15 and angular elements 57 to be kept in stock per implant size can hereby be kept manageable.

The trial plates 17 are provided with a flattened section 18, which simplifies the introduction into the disk space, in the region of their posterior front edge facing cranial or caudal. A bore for the adjustment cylinder 29 of the depth gage 27 extending in the AP direction and a bore 59 for the guide pin 33 not shown in FIG. 5 extending perpendicular thereto are formed in the section of the angular elements 57 extending perpendicular to the AP direction. The guide pin 33 is screwed into the bore 59 and is provided with a slot for this purpose (FIG. 2).

Whereas the determination of the implant size and of the implant inclination takes place preoperatively and results in the selection of the suitable trial plates 17, the determination of the suitable implant height or implant thickness takes place intraoperatively with trial plates 17 introduced into the disk space. For this purpose, a set of trial bodies 63 is provided which have different thicknesses, are attached in each case to a holding bar 65 and which are introduced between the trial plates 17 accordingly pressed apart from one another during the operation. The suitable implant height has been found when the intraoperatively controllable shape of the spine of the patient or the position of the respective bodies of the vertebra best corresponds to the natural shape.

The selection of the implant height by means of the trial bodies 63, and thus the final selection of the implant to be inserted, takes place not only while taking account of the angular presetting determined preoperatively and “simulated” by the inclined trial plates 17, but also while taking account of the insertion depth of the implant likewise determined preoperatively and ideal for the respective patient, i.e. its AP position, which is set at the trial unit 15 by means of the depth gages 27 as a reference for the following steps of the operation. The observation of the preoperatively selected AP position of the implant is therefore ensured by the depth gages 27 in accordance with the invention.

As FIG. 6 shows, the abutment plates 37 each extend up to the section of the angular element 57 extending in the AP direction. The trial plates 17 thus form a posterior end abutment for the abutment plates 37, with this, however, not being absolutely necessary, but alternatively or additionally an end abutment of this type could e.g. also be achieved by an anterior end abutment of the adjustment groove 31.

Coupling sections 61 which project to anterior are made at the sections of the angular elements 57 extending perpendicular to the AP direction for the pressing apart of the trial plates 17. The two jaws of traction forceps are pushed onto the coupling sections 61. The coupling sections 61 are matched to the shape of the forceps for this purpose. Known, commercially freely available traction forces can be used.

The coupling sections 61 are arranged laterally offset from the center so that the forceps are not in the way on the insertion of the trial bodies 63 by means of the holding bars 65.

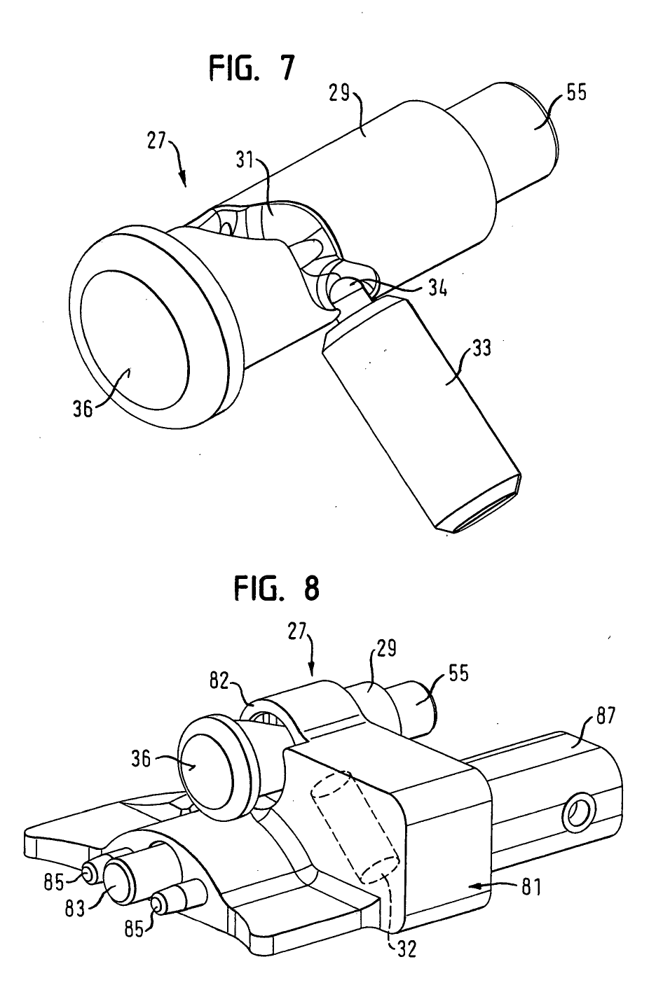

The depth gage 27 shown in FIG. 7 does not have an abutment plate. The end face 36 of the plate-shaped head of the cylindrical adjustment section 29 facing posterior serves as an abutment surface 36. A further difference to the embodiment of FIGS. 1 to 6 consists in the shape of the adjustment groove 31 for the projection 34 formed at the guide pin 33.

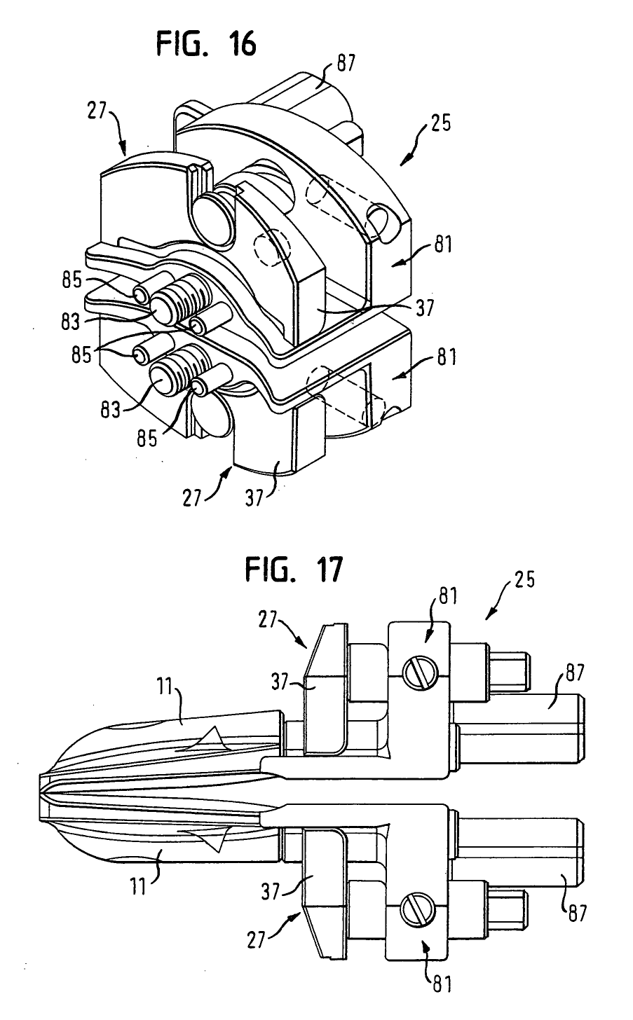

FIG. 8 shows the depth gage 27 at one half of a holding unit. The basic structure of a holding unit in accordance with the invention will be looked at in detail in the following in connection with FIGS. 16 and 17. In contrast to the embodiment shown there, the two halves of the holding unit in accordance with FIG. 8 are provided with a ring section 82 for the leading through of the adjustment section 29 of the depth gage 27. The ring section 82 is formed at an angular element 81 which is moreover provided with a holding screw 83 and positioning pins 85 for the holding of the implant plates not shown here and with a coupling section for a traction instrument. This will be looked at in more detail in connection with FIGS. 16 and 17.

The depth gage 27 is shown in FIG. 8 without the guide pin 33 (FIG. 7). A bore 32 formed in the angular element 81 for the guide pin 33 is indicated by dashed lines in FIG. 8.

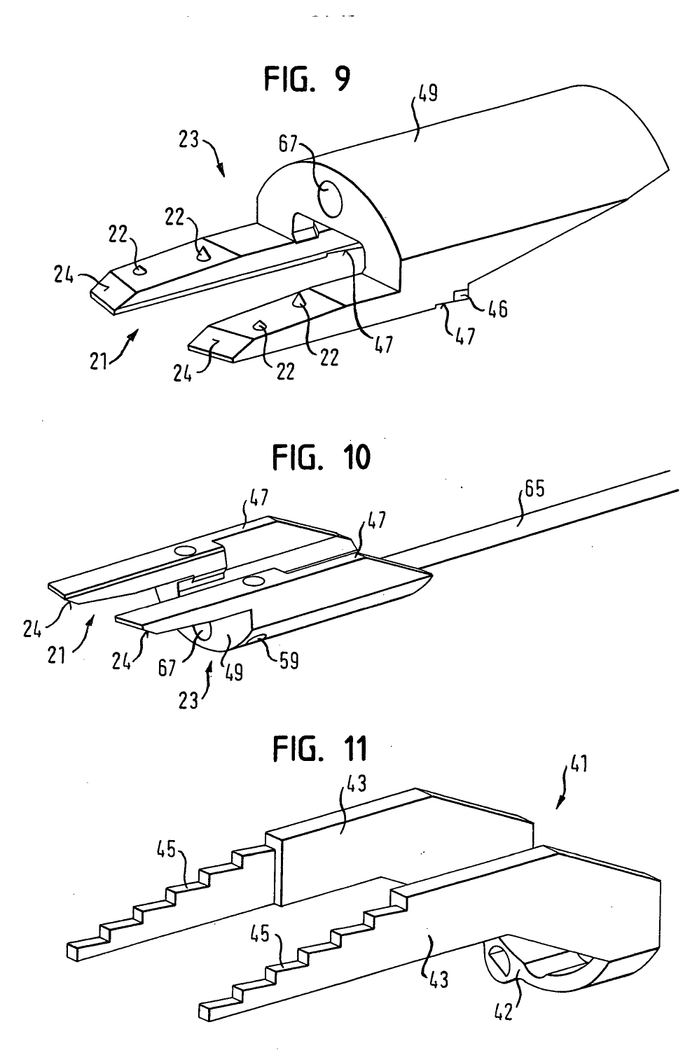

FIGS. 9 to 13 show a machining unit 19 in accordance with the invention which is used for the preparation of the surfaces of the bodies of the vertebra as soon as the suitable implant size is determined.

The machining unit 19 includes two differently formed machining elements 23. The operating element 23 shown in FIG. 9 is also called an upper or cranial operating element in the following and the operating element 23 in accordance with FIG. 10 is also called a lower or caudal operating element although the machining unit 19 can basically be used in the reverse orientation.

The operating elements 23 each include a half-shell like or trough-shaped body section 49 from which a fork-like section 21 with two fork arms extending in the AP direction projects to posterior. To facilitate the introduction of the fork sections 21 into the intervertebral space of the patient, the free ends of the fork arms are each provided with a flattened section 24. To provide the machining unit 19 with a stability on the surfaces of the bodies of the vertebra which is as secure as possible during the machining process, the fork arms are each provided with holding prolongations 22 at the corresponding side.

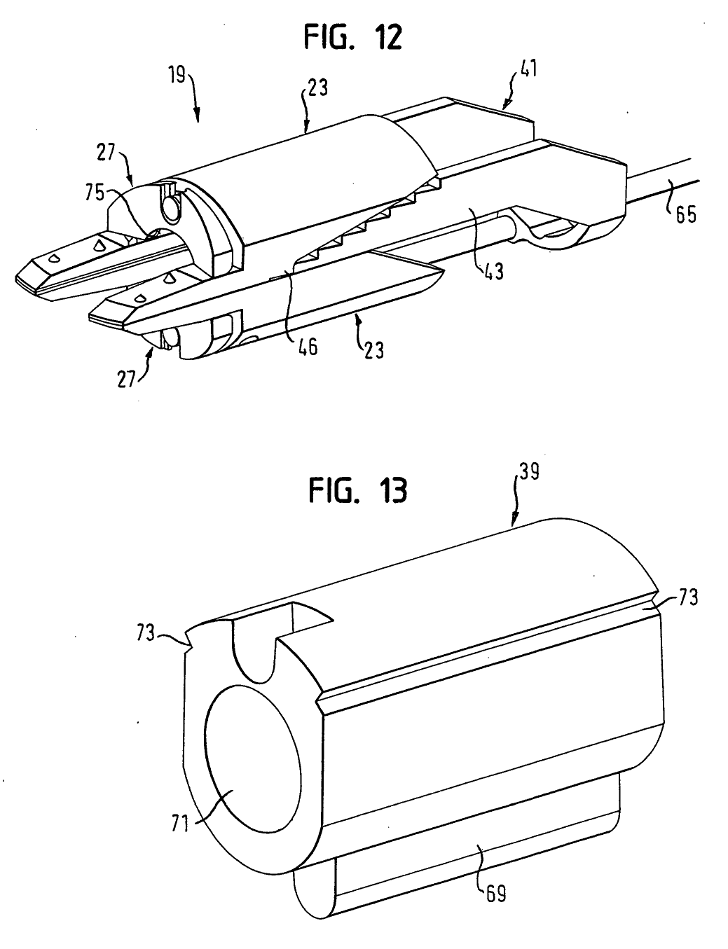

For the attachment of the depth gage 27 in accordance with the invention (FIG. 12), the two operating elements 23 are each provided with a bore 67 for the adjustment cylinder 29 of the depth gage 27 and with a bore 59 (not shown in FIG. 9) for the guide pin 33.

At their open side, the operating elements 23 each have longitudinal edge sections 47 via which the operating elements 23 cooperate with a latching element 41 (FIG. 11) explained in more detail in the following. This longitudinal edge section 47 is formed at a step 46 at the cranial operating element 23 (FIG. 9).

The operating elements 23 are supplied to the intervertebral space during the operation by means of holding bars 65. A holding bar 65 of this type is shown in FIG. 10 in connection with the lower or caudal operating element 23.

The latching element 41 in accordance with the invention in accordance with FIG. 11 includes a U-shaped body section 42 with which it can be guided along the outer sides of the operating elements 23 in the AP direction. The U-limbs of the body section 42 diverge to form latching arms 43 which extend in the AP direction and are provided at their longitudinal edges facing the open side of the body section 42 with a stepped or stair-shaped vertical section 45.

In the assembled state in accordance with FIG. 12, the operating elements 23 are each provided with a depth gage 27. The reference setting of the depth gages 27 determined using the trial unit 15 (FIG. 5) is set accordingly at the depth gages 27 of the machining unit 19 before the latter is introduced with the fork-like operating sections 21 into the intervertebral space. In this connection, the latching element 41 is already located in the starting position shown in FIG. 12 in which the latching arms 43 lie on the longitudinal edge sections 47 of the lower operating element 23 at the end face and contact the steps 46 of the upper operating element 23.

The two half-shell shaped or trough-shaped body sections 49 of the operating elements 23 form a tunnel-like receiving space. A guide sleeve 39 (FIG. 13) for a machining instrument (not shown), in particular in the form of a cutter, is introduced between the latching arms 43 into this receiving space. The guide sleeve 39 is provided on one side with a pair of guide grooves 73 with which guide elements (not shown) formed both at the inner side of the upper operating element 23 and on the inner side of the lower operating element 23 are associated.

The cutter sleeve 39 is held movably by this guidance on the introduction into the receiving space formed by the two operating elements 23 only along a machining axis pre-determined by the guidance relative to the respective operating element 23 and is held immovably at the operating element 23 perpendicular to this machining axis. The direction of the machining axis can, but does not have to, coincide with the AP direction. It is rather preferred for the machining axis pre-determined by the guidance to include a machining angle with the AP direction which corresponds to the angle of inclination of the trial plates 17 or of the implant plates 11. Since this machining angle is comparatively small, the machining axis also still extends approximately in the AP direction in this case.

A clearly set height of the central axis of a passage bore 71 provided for the cutter relative to the fork section 21, and thus to the respective body of the vertebra is predetermined by the guidance of the cutting sleeve 39 perpendicular to the machining axis, in particular therefore approximately in the traction direction or spreading apart direction.

The machining of the respective surface of the body of the vertebra by means of the cutter guided through the bore 71 thus takes place at a height clearly pre-determined by the traction. The machining depth is set by the depth gages 27 previously set to the reference position, said depth gages in turn being matched to a reference for the introduction depth of the sleeve 39 or of the cutter. The operating elements 23 or the sleeve 39 can have end abutments for the cutter.

All parameters of the machining of the bodies of the vertebra are thus already fixed before the introduction of the cutter into the guide sleeve 39, which substantially simplifies the preparation of the surfaces of the bodies of the vertebra for the surgeon.

In contrast to the depth gages 27 used in connection with the trial unit 15 (FIG. 5), the abutment plates 38 of the depth gages 27 used in connection with the machining unit 19 (FIG. 12) are each provided with a cut-out 75 for the cutter guided through the bore 71 of the guide sleeve 39.

To machine the other body of the vertebra subsequent to the preparation of the one surface of the body of the vertebra, the guide sleeve 39 only has to be pulled out of the receiving space, rotated about 180° around an axis extending in the AP direction and pushed onto the other operating element 23.

The machining of the bodies of the vertebra by means of the cutter guided by the sleeve 39 takes place at a spacing between the two operating elements 23 set with the help of the traction forceps. For this purpose, adapter elements (not shown) are provided which are pushed onto the jaws of the traction forceps to match the traction forceps to engagement regions (not shown) at the inner sides of the operating elements 23 formed in accordance with the adapter elements. The adapter elements are made in a self-latching manner in that they are provided with special undercuts which reliably prevent an accidental release of the traction forceps from the operating elements 23 in the state applied to the operating elements 23. Separate latching devices are hereby not necessary.

During the spreading apart procedure, the latching element 41 is pushed step-wise further between the operating elements 23. The straight longitudinal edges of the latching arms 43 disposed opposite the stepped vertical profiles 45 lie on the longitudinal edge sections 47 of the lower operating element 23, whereas the steps 46 formed at the upper operating element 23 cooperate with the stair sections 45 of the latching arms 43. Consequently, a traction height once achieved can be secured by a step-wise insertion of the latching element 41, i.e. the machining unit 19 can be latched in this position. As soon as the desired traction height determined in connection with the trial unit 15 (FIG. 5) and corresponding to the suitable trial body 63 (FIG. 6) has been reached and secured by means of the latching element 41, the traction forceps—including the adapter elements—can be removed. The tunnel formed by the operating elements 23 is now free to receive the guide sleeve 39.

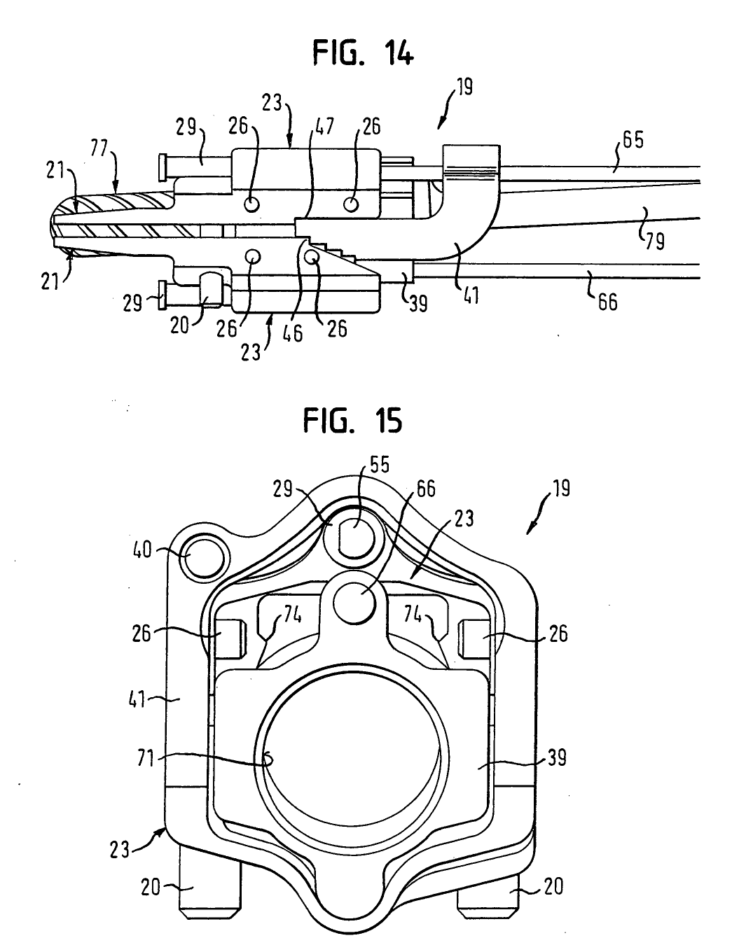

The further embodiment of a machining unit 19 in accordance with the invention shown in FIGS. 14 and 15 includes two operating elements 23 which are each provided with a depth gage in accordance with FIG. 7 of which the adjustment cylinder 29 and (FIG. 15) the actuation section 55 are shown. The operating elements 23 are guided relative to one another by pins 20 in the spreading apart direction, i.e. perpendicular to the AP direction.

Furthermore, a machining instrument 77—omitted in FIG. 15—is shown in FIG. 14 in the form of a cutter which is guided by the guide sleeve 39 coupled to the upper operating element 23 in FIG. 14 and to the lower operating element 23 in FIG. 15. The previously already mentioned machining angle different to zero between the holding bar 79 and the axis of rotation of the cutter and the theoretical AP direction can in particular be seen from FIG. 14, with the holding bar 65 of the upper operating element 23 shown in FIG. 14 extending parallel to said AP direction. When the cutter 77 is inserted, a holding bar 66 of the guide sleeve 39 extends at an inclination to its holding bar 79.

The latching element 41 cooperating with the steps 46 or with the longitudinal edge sections 47 of the operating elements 23 can be handled via a holding bar (not shown) for which it is provided with an opening 40 (FIG. 15) arranged offset laterally outwardly.

The guide sleeve 39 provided with the bore 71 for the cutter 77 can first be coupled to the one operating element 23 and then with the other operating element 23 in order to machine the two bodies of the vertebra successively. For this purpose, the operating elements 23 are provided with guide pins 26, only recognizable at the upper operating element 23 in FIG. 15, for corresponding guide grooves of the guide sleeve 39. Furthermore, the operating elements 23 are provided with a channel-like recess for the guidance of the correspondingly shaped side of the guide sleeve 39 disposed opposite the section for the holding bar 66.

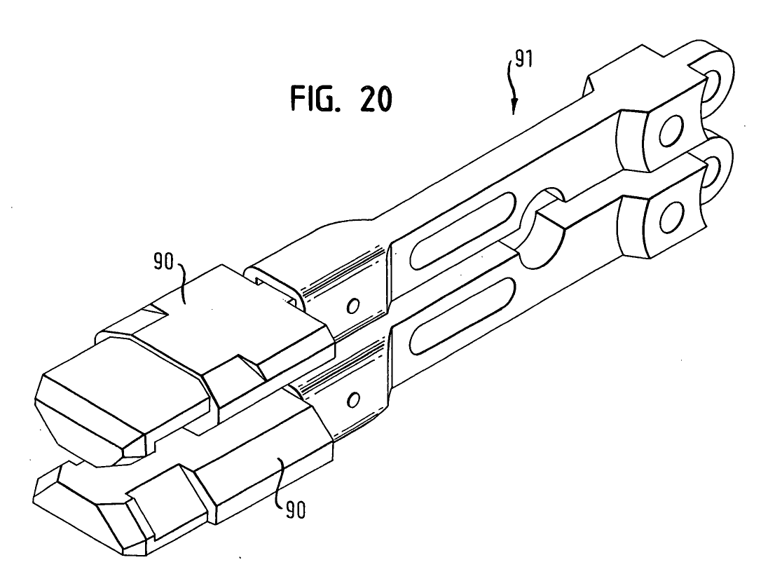

Furthermore, the operating elements 23 are each provided with inwardly disposed shoulders 74 via which the operating elements 23 can be supported at adapter or extension elements 90 shown in FIG. 20 which are pushed onto the front ends of traction forceps 91.

The holding unit 25 shown in FIGS. 16 and 17 corresponds to the trial unit 15 with respect to its basic design (FIG. 5). The sections of the angular elements 81 extending in the AP direction are each provided with positioning pins 85 and with a passage bore disposed between the pins 85 for a separate holding screw 83. The respective implant plate 11 (FIG. 17) is fixedly connected to the angular element 81 by means of the holding screws 83 on the insertion into the intervertebral space in the position relative to the angular element 81 set by the positioning pins 85. The implant plates 11 are each provided at their anterior rear sides with corresponding insertion openings for the positioning pins 85 and with a threaded bore for the holding screw 83.

Instead of the purely cylindrical positioning pins 85 shown in FIG. 16, positioning pins with differently shaped free ends can also be used. For example, the free end sections of the positioning pins 85 can converge in a sharply conical manner or be made in a frusto-conical manner, with the length of these non-cylindrical end sections of the pins also being able to vary. Alternatively to this pin/hole positioning, a so-called seated positioning can also be provided in which one or more support projections can be arranged in spade-like form, for example, instead of the pins 85, with the implant plates 11 secured by means of the holding screws 83 being seated on them. A secure positioning of the implant plates 11 to be inserted can also be achieved in this manner.

The cross-sectional surfaces of the sections of the angular elements 81 extending parallel to the AP direction are each shaped in accordance with the anterior rear side of the implant plate 11 and accordingly provided with a central convex elevated section through which the passage bore for the holding screw 83 extends. The abutment plates 37 of the depth gages 27 are therefore provided with a corresponding cut-out.

FIG. 13 shows the holding unit 25 in accordance with the invention with implant plates 11 attached thereto and fixed by means of the holding screws 83 (FIG. 16). The correct insertion depth corresponding to the implant selection for the implant plates 11 is in turn ensured by the depth gages 27 in accordance with the invention at which the reference position previously determined with the depth gages 27 of the trial unit (FIG. 5) is set.

FIG. 17 shows the optimum case in which the depth gages 27 are set such that the rear anterior edge of the implant plates 11 lies approximately 4 mm behind—seen from anterior to posterior—the anterior edge (not shown) of the bodies of the vertebra. Depending on the setting of the depth gages 27, i.e. in dependence on the spacing between the abutment plates 37 and the sections of the angular elements 81 extending perpendicular to the AP direction, the anterior edge of the implant plates 11 is disposed more or less far from the anterior edge of the body of the vertebra.

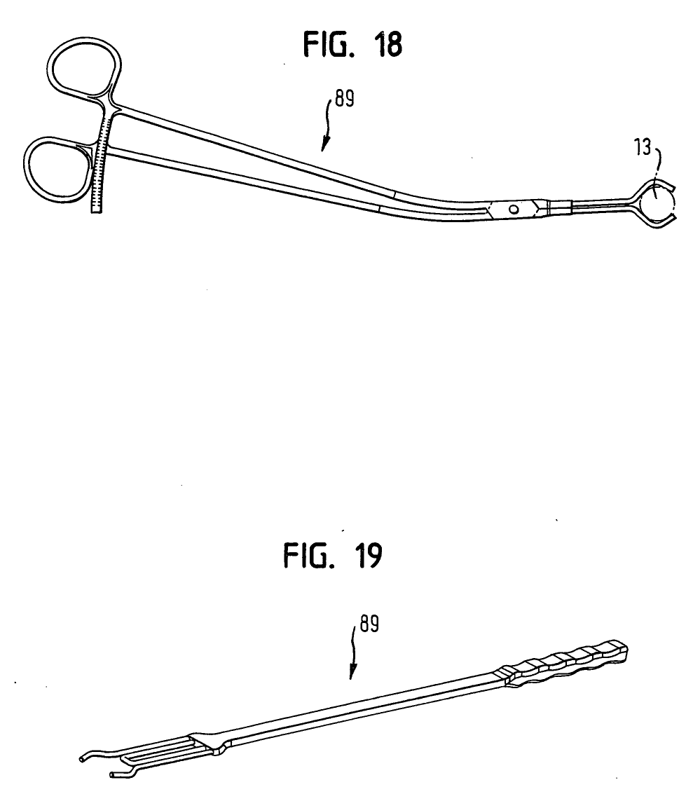

The angular elements 81 are coupled to the traction forceps via coupling sections 87 arranged offset from the center corresponding to the trial unit 15 (FIG. 5). As soon as the implant plates 11 introduced into the intervertebral space are at the desired depth and the bodies of the vertebra have been spread apart sufficiently widely by actuation of the traction forceps, the matching implant core 13 (FIG. 18), preferably made in lens-shape and which will not be looked at in any more detail, is brought between the implant plates 11. Holding forceps 89 are provided for this purpose. Holding forceps 89 of the so-called crab type are shown in FIG. 18. Alternatively, holding forceps of the so-called needle-holder type can, for example, also be used such as are shown by way of example in FIG. 19.

As soon as the implant core 13, also called an inlay, is located in the desired position between the two tractioned implant plates 11, the traction is terminated, i.e. the traction forceps are so-to-say “released”. The intervertebral disk implant consisting of the two implant plates 11 and the implant core 13 thus comes into its end configuration. To release the angular sections 81 of the holding unit 25 from the implant plates 11, the holding screws 83 are released by means of a screwdriver and the angular sections 81 are withdrawn together with the positioning pins 85. The holding unit 25 is subsequently moved out. The holding screws 83 are accessible from the anterior side by means of the screwdriver, with the arrangement of the coupling sections 87 for the traction forceps offset laterally from the center not only facilitating the insertion of the implant core 13 by means of the holding forceps 89, but also the release of the holding screws 83.

The course of an operation carried out by means of the instrument system in accordance with the invention for the insertion of an intervertebral disk implant including two implant plates 11 and an implant core 13 already results form the above description of the set of instruments in accordance with the invention. Special aspects of the invention will be looked at in the following and the concept in accordance with the invention will again be shown in context.

As already mentioned above, in the first phase of the operation, the preoperative phase, the size of the implant and the angle of inclination of the implant plates 11 are first determined, that is the angle of inclination of the sides of the implant plates 11 facing the surfaces of the vertebral bodies in the inserted state relative to the AP direction. Furthermore, the optimum position of the implant in the AP direction, that is the insertion depth, is determined in this preoperative phase. This preoperative setting of surgical parameters is carried out with the help of photographic material which can be obtained by different methods which are generally known (e.g. X-ray, MRI, CT), and which should not be looked at in more detail at this point.

For the subsequent intraoperative parameter determination, the previously determined AP position is set by means of a special screwdriver at the depth gages 27 of the trial unit 15 (FIG. 5). A trial unit 15 is used whose trial plates 17 correspond to the pre-selected implant both with respect to the preoperatively determined implant size and with respect to the preoperatively determined angle of inclination. However, a procedure is followed such that one approaches the preoperatively determined position in a step-wise manner using monitor control.

The special screwdriver with which the AP setting is carried out at the depth gages 27 is characterized by a display device at which the carried out AP setting can be read off and so be used as a reference for the following AP settings. For this purpose, in a preferred embodiment, the screwdriver can be provided with a scale plate which is arranged rotationally fixedly with the screwdriver shaft in the proximity of the handle. A clear, relative rotational position is present between the screwdriver shaft and the adjustment cylinder 29 of the depth gage 27 by the design of the actuation sections 55 (FIG. 4, FIG. 5 and FIG. 6) of the depth gages 27 differing from a purely circular cylindrical shape. The AP setting carried out at the trial unit 15 can consequently be read off at the screwdriver and be correctly transferred to the depth gages 27 of the machining unit 19 and of the holding unit 25.

The traction forceps used in the selection of the suitable trial body 63 (FIG. 6) for the spreading apart of the trial unit 15 likewise have the possibility of reading off a traction height selected as suitable and of being set again reproducibly in subsequent traction procedures. Subsequent to the intraoperatively carried out determination of the suitable implant height by means of the trial bodies 63, the surgeon consequently has both a—preoperatively determined—reference value for the AP setting and a reference value—determined intraoperatively by means of the trial unit 15—for the traction height.

The subsequent preparation of the surfaces of the bodies of the vertebra by means of the machining unit 19 (FIGS. 12 and 13) thus takes place in a position of the decisive guide sleeve 39 serving for the guidance of the cutter instrument relative to the body of the vertebra in question which is clearly set by the AP setting at the depth gages 27 of the machining unit 19 and by the previously determined traction height. The machining of the bodies of the vertebra is consequently carried out in a manner directly matched to the selected implant and extremely comfortable for the surgeon.

Subsequent to the preparation of the surfaces of the bodies of the vertebra, the implant plates 11 of the implant are inserted by means of the holding unit 25, with the depth gages 27 set in accordance with the previously determined reference value in turn ensuring the correct AP position and thus the correct insertion depth of the implant plates 11 and thus of the implant in total.

The surgeon is provided by the invention with a simply usable set of instruments including a manageable number of individual parts with which intervertebral disk operations can be carried out with a high degree of precision and safety.

The description of the invention is merely exemplary in nature and, thus, variations that do not depart from the gist of the invention are intended to be within the scope of the invention. Such variations are not to be regarded as a departure from the spirit and scope of the invention.

### REFERENCE NUMERAL LIST

11 implant plate
13 implant core
15 trial unit
17 trial section, trial plate
18 flattened area
19 machining unit
20 pin
21 fork-like section of the operating element
22 holding projection
23 operating element
24 flattened area
25 holding unit
26 guide pin
27 depth gage
29 adjustment section of the depth gage, adjustment cylinder
31 adjustment groove
32 bore
33 guide pin
34 projection
35 latch recess
36 abutment surface
37 abutment plate
38 abutment surface
39 guide sleeve
40 opening
41 latching element
42 body section
43 latching arm
45 vertical section
46 step
47 longitudinal edge section
49 body section
51 cut-out of the abutment plate
52 latching section
53 latching head
55 actuation section
57 angular element
59 bore
61 coupling section
63 trial body
65 holding bar
66 holding bar
67 bore for depth gage
69 holding section
71 bore
73 guide groove
74 shoulder
75 cut-out
77 machining instrument
79 holding bar
81 angular element
82 ring section
83 holding screw
85 positioning pin
87 coupling section
89 holding forceps
90 extension element
91 traction instrument

What is claimed is:

1. An instrument system for the insertion of intervertebral disk implants including two implant plates (11) and one implant core (13), comprising:
a trial unit (15) including two plate-shaped trial sections (17) for determining a particular implant that is suitable for a respective patient;
a machining unit (19) that prepares surfaces of bodies of vertebra of the patient and that includes operating elements (23) having two fork-like sections (21); and
a holding unit (25) that can be coupled to the implant plates (11) for insertion of the implant,
wherein the trial unit (15), the machining unit (19) and the holding unit (25) can each be coupled to a traction instrument for pressing apart the trial sections (17), the operating elements (23) and the implant plates (11), respectively.

2. An instrument in accordance with claim 1, wherein the traction instrument is a forceps type traction instrument.

3. An instrument system in accordance with claim 1, wherein the trial unit (15), the machining unit (19) and the holding unit (25) are each provided with coupling sections (61) for the traction instrument at respective rear sides that are remote from a surgical area during surgery.

4. An instrument system in accordance with claim 1, wherein the trial unit (15) has an adjustable depth gage (27) for fixing a reference position for the implant in an anterior-posterior direction.

5. An instrument system in accordance with claim 4, wherein at least one of the machining unit (19) and the holding unit (25) include an adjustable depth gage (27), which is adjustable in accordance with the reference position of the trial unit (15).

6. An instrument system in accordance with claim 5, wherein the depth gages (27) are made in at least substantially the same construction.

7. An instrument system in accordance with claim 4, wherein the depth gage (27) includes a caudal and a cranial section which can be set independently of one another.

8. An instrument system in accordance with claim 4, wherein the depth gage (27) is made at least regionally in the manner of an adjusting screw adjustable relative to the trial unit (15) in the anterior-posterior direction.

9. An instrument system in accordance with claim 4, wherein the depth gage (27) is coupled to the trial unit (15) via a pin and groove guide.

10. An instrument system in accordance with claim 4, wherein the depth gage (27) has an adjustment section (29) that is rotatable by means of a screwdriver about an axis extending substantially parallel to the anterior-posterior direction, at which an adjustment groove (31), which extends at least regionally in the manner of a helix or a screw around the axis of rotation, is made for a guide pin (33) arranged at the trial unit (15).

11. An instrument system in accordance with claim 10, wherein the adjustment groove (31) has a plurality of latch recesses (35) for the guide pin (33) which are spaced apart from one another and correspond to pre-determined depth positions.

12. An instrument system in accordance with claim 4, wherein the depth gage (27) has an abutment plate (37) that extends substantially perpendicular to the anterior-posterior direction and fixes the position of the trial unit (15) relative to the body of the vertebra during the operation.

13. An instrument system in accordance with claim 12, characterized in that an adjustment section (29) of the depth gage (27) fixedly connected to the abutment plate (37) in the anterior-posterior direction is rotatable relative to the abutment plate (37).

14. An instrument system in accordance with claim 1, wherein the machining unit (19) includes at least one guide sleeve (39) for a machining instrument, the guide sleeve (39) being able to be coupled to the machining unit (19) for the fixing of a machining position, whereby the guide sleeve (39) is fixedly held with respect to the machining unit (19) in directions perpendicular to a machining axis extending in or inclined to the anterior-posterior direction, with a machining angle between the machining axis and the anterior-posterior direction corresponding to an angle of inclination of the implant plates (11) which lies in the range from 2° to 7°.

15. An instrument system in accordance with claim 14, wherein the machining instrument includes one of a cutter and a drill.

16. An instrument system in accordance with claim 14, wherein the guide sleeve (39) is made both for caudal and for cranial machining and can be coupled for this purpose first with the one operating element (23) and then with the other operating element (23).

17. An instrument system in accordance with claim 14, wherein the machining unit (19) regionally bounds a tunnel-like receiving space into which the guide sleeve (39) can be inserted at least partly in the anterior-posterior direction.

18. An instrument system in accordance with claim 1, wherein the machining unit (19) includes a separate latching element (41) with which a spacing between the operating elements (23) established by means of the traction instrument can be fixed.

19. An instrument system in accordance with claim 18, wherein the latching element (41) has at least one latching section (43) that is pushable between the operating elements (23) in the anterior-posterior direction and that has a stepped vertical section (45) extending in a stair-like manner.

20. An instrument system in accordance with claim 18, wherein the latching section (43) is fork-shaped and includes two latching arms (43) which have identical vertical sections (45) and which can each be pushed between longitudinal edge sections (47) facing one another of the operating elements (23) constructed as one of half-shells and troughs in this region.
